(19) United States
(12) Patent Application Publication
(10) Pub. No.: US 2023/0248369 A1
(43) Pub. Date: Aug. 10, 2023

(54) DEVICES AND METHODS FOR DELIVERING AGENTS

(71) Applicant: Boston Scientific Scimed Inc., Maple Grove, MN (US)

(72) Inventors: Andrew PIC, Northboro, MA (US); Ciara MURPHY, Pepperell, MA (US)

(73) Assignee: Boston Scientific Scimed Inc., Maple Grove, MN (US)

(21) Appl. No.: 18/161,971

(22) Filed: Jan. 31, 2023

Related U.S. Application Data
(60) Provisional application No. 63/307,947, filed on Feb. 8, 2022.

(51) Int. Cl. A61B 17/12

(52) U.S. Cl. CPC A61B 17/12186; A61B 2017/00681; A61B 2017/1205; A61B 2017/12004

(57) ABSTRACT

A device for delivering an agent that includes a housing configured to store an agent and a cone assembly at least partially received within the housing. The cone assembly including an inner wall configured to be adjacent to the agent, an outlet channel extending from the inner wall, an inlet channel, and an intermediate channel between the outlet channel and the inlet channel. The inlet channel is in fluid communication with the outlet channel via the intermediate channel. The device includes a filter disposed within the inlet channel and positioned adjacent to the intermediate channel. The filter including a plurality of pores configured to permit a fluid received by the inlet channel to pass through the intermediate channel to mix with the agent adjacent to the inner wall.

### CROSS REFERENCE TO RELATED APPLICATIONS

This application claims the benefit of priority of U.S. Provisional Pat. Application No. 63/307,947, filed Feb. 8, 2022, the entirety of which is incorporated herein by reference.

### TECHNICAL FIELD

Various aspects of this disclosure relate generally to devices and methods for delivering agents. More specifically, in embodiments, this disclosure relates to devices for delivery of powdered agents, such as hemostatic agents.

### BACKGROUND

In certain medical procedures, it may be necessary to minimize or stop bleeding internal to the body. For example, an endoscopic medical procedure may require hemostasis of bleeding tissue within the gastrointestinal tract, for example in the esophagus, stomach, or intestines.

During an endoscopic procedure, a user inserts a sheath of an endoscope into a body lumen of a patient. The user utilizes a handle of the endoscope to control the endoscope during the procedure. Tools are passed through a working channel of the endoscope via, for example, a port in the handle, to deliver treatment at the procedure site near a distal end of the endoscope. The procedure site is remote from the operator.

To achieve hemostasis at the remote site, a hemostatic agent may be delivered by a device inserted into the working channel of the endoscope. Agent delivery may be achieved through mechanical systems, for example. Such systems, however, may require numerous steps or actuations to achieve delivery, may not achieve a desired rate of agent delivery or a desired dosage of agent, may result in the agent clogging portions of the delivery device, may result in inconsistent dosing of agent, or may not result in the agent reaching the treatment site deep within the GI tract. The current disclosure may solve one or more of these issues or other issues in the art.

### SUMMARY

Each of the aspects disclosed herein may include one or more of the features described in connection with any of the other disclosed aspects.

According to an example, a device for delivering an agent may include a housing configured to store an agent; a cone assembly at least partially received within the housing, the cone assembly including: an inner wall configured to be adjacent to the agent; an outlet channel extending from the inner wall; an inlet channel; and an intermediate channel between the outlet channel and the inlet channel, such that the inlet channel is in fluid communication with the outlet channel via the intermediate channel; a filter disposed within the inlet channel and positioned adjacent to the intermediate channel, the filter including a plurality of pores configured to permit a fluid received by the inlet channel to pass through the intermediate channel to mix with the agent adjacent to the inner wall.

Any of the devices described herein may include any of the following features. The outlet channel is coupled to an outlet tube, such that the outlet tube is in fluid communication with the agent via the outlet channel, and the inlet channel is coupled to an inlet tube, such that the inlet tube is in fluid communication with the agent via the inlet channel. The inlet tube is configured to supply the fluid to the intermediate channel and through the filter for mixing with the agent. The outlet tube is configured to receive the agent from the inner wall via the outlet channel when the agent is moved by the fluid supplied from the inlet tube. The outlet tube includes a first end disposed within the outlet channel and positioned relatively underneath the intermediate channel. The outlet tube includes a first end disposed within the outlet channel and positioned relative to the intermediate channel such that the first end overlaps with the intermediate channel. The filter includes an insert received within the inlet channel. The filter is sintered, includes a porous metal, or includes a lattice printed material. The plurality of pores extend along tortuous paths between opposing ends of the filter. The cone assembly includes an opening at a first end of the cone assembly, and a diameter of space defined by an exterior surface of the inner wall decreases in a direction toward the opening at the first end. The intermediate channel is positioned relatively underneath the opening, and extends at least partially within the outlet channel and the inlet channel. The filter is coupled to the cone assembly within the inlet channel, such that the filter is removable from the inlet channel. The filter is integrally formed with the cone assembly. The housing includes a first body and a second body that are configured to engage the cone assembly along an outer rim of the cone assembly. The agent is a powder having a particle diameter between 320 microns and 500 microns, and the plurality of pores have a diameter between 40 microns and 100 microns.

According to another example, a device for delivering an agent may include a housing configured to store an agent; a cone assembly coupled to the housing, and configured to receive the agent, wherein the cone assembly includes a wall configured to be adjacent to the agent, and an intermediate channel positioned relatively below the wall at an end of the wall opposite an end of the wall that receives the agent, for controlling a flow of fluid toward the agent; and a filter coupled to the cone assembly alongside the intermediate channel, the filter includes a plurality of pores configured such that the flow of fluid is allowed to pass through the intermediate channel via the filter and into a cavity defined by an inner surface of the wall to agitate the agent within the cone assembly.

Any of the devices described herein may include any of the following features. The filter is positioned such that the flow of fluid is permitted to pass through the intermediate channel only after passing through the plurality of pores, and the plurality of pores are configured such that the agent is not permitted to pass through the filter. The housing includes a first body and a second body that are configured to engage an outer rim of the cone assembly to securely couple the cone assembly to the housing. The filter is sintered, includes a porous metal, or includes a lattice printed material.

According to another example, a device for delivering an agent may include a housing configured to hold an agent; a cone coupled to the housing for funneling the agent toward an opening, the cone includes an outlet in fluid communication with the opening, an inlet in fluid communication with the outlet, and an intermediate channel between the outlet and the inlet; and a sintered filter disposed within the inlet and positioned adjacent to the intermediate channel, the sintered filter including a plurality of pores that are configured such that gas is permitted to pass through the filter and into the cone via the intermediate channel to move the agent within the housing, wherein the intermediate channel is sized to control a flow of the gas into the cone for moving the agent.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “top” refers to a direction or side of a device relative to its orientation during use, and the term “bottom” refers to a direction or side of a device relative to its orientation during use that is opposite of the “top.” The term “exemplary” is used in the sense of “example,” rather than “ideal.” The term “approximately,” or like terms (e.g., “substantially”), includes values +/- 10% of a stated value.

### BRIEF DESCRIPTION OF THE DRAWINGS

The accompanying drawings, which are incorporated in and constitute a part of this specification, illustrate aspects of this disclosure and together with the description, serve to explain the principles of the disclosure.

FIG. 1 shows an exemplary delivery device.

FIG. 2 shows a side view of an exemplary dispensing portion having a cone assembly for the delivery device of FIG. 1.

FIG. 3 shows a cross-sectional view of the dispensing portion of FIG. 2, taken along line 3-3 in FIG. 2.

FIG. 4 shows a cross-sectional side view of the dispensing portion of FIG. 2.

FIG. 5 shows a partial side view of the dispensing portion of FIG. 2.

FIG. 6 shows a side view of the cone assembly of FIG. 2.

FIG. 7 shows a cross-sectional view of the cone assembly of FIG. 2, taken along line 7-7 of FIG. 6.

### DETAILED DESCRIPTION

Embodiments of this disclosure relate to dispensing portions having cone assemblies for storing an agent (e.g., a powdered agent) and mechanisms for delivering the agent to a site of a medical procedure. The cone assembly may house a porous filter through which a pressurized fluid may pass prior to encountering the agent. The agent may be received within an enclosure of the dispending portion and in fluid communication with the pressurized fluid through the porous filter. Accordingly, when the pressurized fluid passes through the porous filter, it may enter the enclosure to agitate the agent prior to its delivery to the site of the medical procedure. Aspects of the dispending portion and cone assembly, such as the enclosure and/or porous filter, may facilitate a fluidization of the agent with the flow of pressurized fluid prior to the agent being delivered, which may assist in preventing or minimizing clogging during delivery.

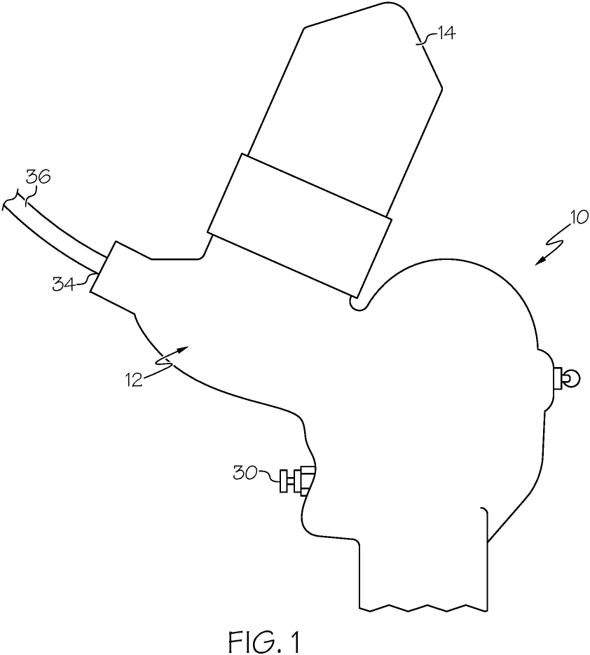

FIG. 1 shows a delivery system 10, which may be a powder delivery system. Delivery system 10 may include a body 12. Body 12 may include, or may be configured to receive, an enclosure 14 (or other source) storing an agent. Enclosure 14 may be coupled to body 12 for providing agent to body 12, or a lid/enclosure of the agent may be screwed onto, or otherwise coupled to, enclosure 14 for supplying the agent to enclosure 14. The agent may be, for example, a powdered agent, such as a hemostatic agent. The agent may alternatively be another type of agent or material or form of agent (e.g., a liquid or gel agent) and may have any desired function. Enclosure 14 may be removably attached to other components of delivery system 10, including components of body 12. Body 12 may have a variety of features, to be discussed in further detail herein. U.S. Pat. Application No. 16/589,633, filed Oct. 1, 2019, the disclosure of which is hereby incorporated by reference in its entirety, discloses features of exemplary delivery devices and systems. The features of this disclosure may be combined with any of the features described in the above-referenced application. The features described herein may be used alone or in combination and are not mutually exclusive. Like reference numbers and/or terminology are used to denote similar structures, when possible.

An actuation mechanism 30 may be used to activate flow of a pressurized fluid (e.g., gas). Fluid alone or a combination of agent and fluid may be delivered from outlet 34 of body 12. Outlet 34 may be in fluid communication with a catheter 36 or other component for delivering the combination of agent and fluid to a desired location within a body lumen of a patient.

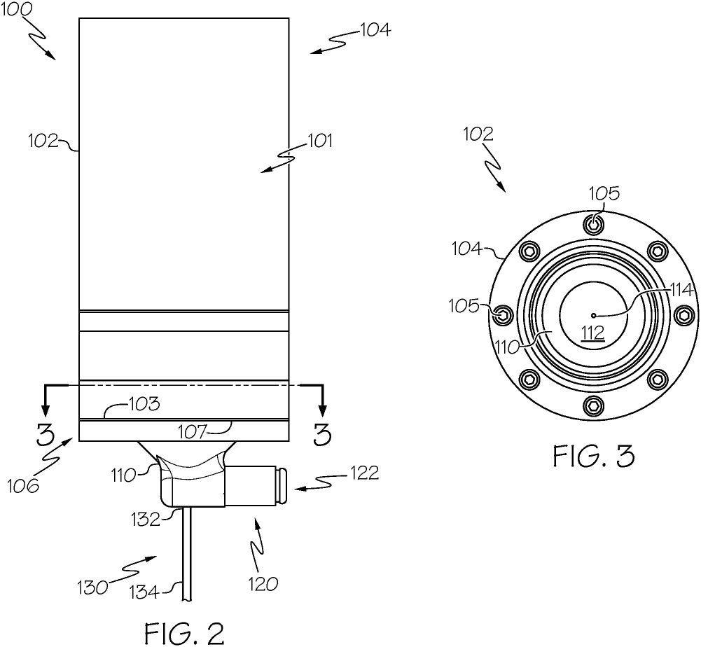

FIG. 2 show aspects of an exemplary dispensing portion 100. Dispensing portion 100 may be used in place of enclosure 14 of delivery system 10. Dispensing portion 100 may include a housing 102 (which may have any of the properties of enclosure 14) that stores an agent. In the embodiment, housing 102 may include a first body 104 and a second body 106 coupled to one another along corresponding ends. For example, first body 104 may include a bottom (second) end 103 that is configured to interface with a top (first) end 107 of second body 106, thereby defining a cavity 101 of housing 102. Each of first body 104 and second body 106 may have inner surfaces defining cavity 101 for storing the agent in housing 102.

Dispensing portion 100 may include an insert received at least partially within housing 102, such as a cone assembly 110 coupled between first body 104 and second body 106. Cone assembly 110 may be positioned relatively below first body 104 and/or second body 106, such that cone assembly 110 may be configured to receive the agent stored in housing 102 (e.g., by gravitational forces) when coupled to the bodies 104, 106. In some embodiments, dispensing portion 100 may include a valving system configured to meter the agent to cone assembly 110.

Cone assembly 110 may be in further fluid communication with a pressurized fluid source (not shown) coupled thereto via an inlet tube, such as a fluid inlet 120. Fluid inlet 120 may include an inlet port 122 that, when disposed within body 12 of delivery system 10, may be fluidly coupled to a channel and/or tube that is in fluid communication with the pressurized fluid source. As described herein, fluid inlet 120 may be configured to transfer a fluid received from the pressurized fluid source into housing 102 via cone assembly 110, thereby causing the agent stored within cavity 101 to move within first body 104 and second body 106.

Although fluid inlet 120 is shown at a side portion of cone assembly 110, it will be appreciated that fluid inlet 120 may be fluidly coupled to dispensing portion 100 in additional and/or alternative locations. For example, fluid inlet 120 may be fluidly coupled to housing 102 in lieu of and/or in addition to cone assembly 110, such as, for example, along first body 104 or second body 106.

Still referring to FIG. 2, dispensing portion 100 may also have an outlet at cone assembly 110. An outlet tube 130 (e.g., a hypotube) may define the outlet of dispensing portion 100, and outlet tube 130 may include a top (first) end 132 coupled to cone assembly 110 and a bottom (second) end 134 that is in fluid communication with outlet 34 of delivery system 10 (see FIG. 1). In some embodiments, top (first) end 132 may be removably coupled to cone assembly 110 via a luer connection, compression fitting, or other suitable means. In other embodiments, top (first) end 132 may be fixedly attached to cone assembly 110. As described herein, dispensing portion 100 may be configured and operable to deliver the agent stored in housing 102 and/or pressurized fluid received from the pressurized fluid source to catheter 36 via outlet tube 130 upon fluidizing the agent in cavity 101.

Referring now to FIG. 3, a bottom portion of first body 104 is shown with cone assembly 110 coupled to housing 102. First body 104 may be coupled to second body 106 via one or more fastening mechanisms 105 (e.g., screws, bolts, etc.) to allow the bottom (second) end 103 of first body 104 to be securely attached to the top (first) end 107 of second body 106. In some embodiments, housing 102 may be selectively assembled and disassembled by actuating the one or more fastening mechanisms 105 to attach and separate first body 104 and second body 106, respectively. In other embodiments, first body 104 and second body 106 may be fixed to one another without a capability of detaching housing 102.

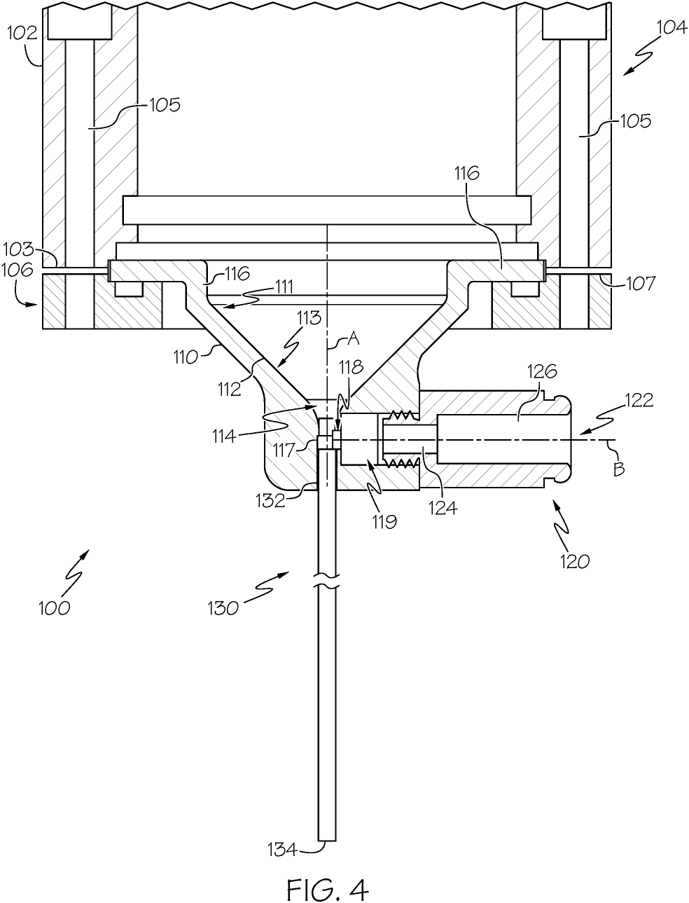

Cone assembly 110 may include an inner wall 112 that extends downward toward second body 106, away from first body 104, and that terminates at an exit opening 114 positioned at a center of inner wall 112. As best seen in FIG. 4, cone assembly 110 may have a longitudinal length that is parallel to a central axis A of cone assembly 110. Further, cone assembly 110 may extend about central axis A, and inner wall 112 may be angled / tapered radially-inward toward central axis A. Exit opening 114, terminating at the center of inner wall 112, may be positioned in alignment with central axis A. Accordingly, inner wall 112 may form a conical or funnel shape about central axis A. An interior surface of inner wall 112 may be shaped such that the interior of cone assembly 110 may have a varying diameter that decreases from a top (first) end 111 of cone assembly 110 (e.g., adjacent to top (first) end 107) to a bottom (second) end 113 of cone assembly 110 (e.g., adjacent to exit opening 114).

In particular, at the first (top) end 111 (the top of inner wall 112), the interior within inner wall 112 may have its greatest diameter. Inner wall 112 may be angled relative to central axis A such that, moving downward toward the bottom (second) end 113, the interior within inner wall 112 may taper to a smaller diameter. In the example, cone assembly 110 may have a height defined between the top and bottom ends of inner wall 112 ranging from about 0.70 inches to about 0.80 inches. Further, the interior of cone assembly 110 may have a first diameter defined at the top end of inner wall 112 ranging from about 0.80 inches to about 0.90 inches. As described below, the interior of cone assembly 110 may have a second diameter at the bottom end of inner wall 112 that corresponds to a diameter of exit opening 114.

As described herein, as the agent moves downward through cone assembly 110, it may encounter portions of inner wall 112 that vary in diameter, which may reduce the agent from clogging within dispensing portion 100. The agent may be prone to bridging, which may result in clogging absent the variations in diameter of cone assembly 110, and particularly the interior space within inner wall 112. In some embodiments, exit opening 114 may have a cone-like shape and varying diameter to further reduce the agent from clogging at exit opening 114 prior to exiting dispensing portion 100 via outlet tube 130. In the example, exit opening 114 may have a diameter ranging from about 0.05 inches to 0.07 inches.

Still referring to FIG. 4, cone assembly 110 may include an outer annular rim 116 extending about the top (first) end 111 of inner wall 112. The outer annular rim 116 may be received between the bottom (second) end 103 of first body 104 and the top (first) end 107 of second body 106. Accordingly, first body 104 and second body 106 may engage outer rim 116, thereby securing cone assembly 110 to housing 102. The outer annular rim 116 may have a diameter ranging from about 1.50 inches to about 1.60 inches.

Cone assembly 110 may further include an outlet channel 117, an intermediate channel 118, and an inlet channel 119 positioned beneath exit opening 114. Outlet channel 117 may be in alignment and in fluid communication with exit opening 114, and configured to receive the top (first) end 132 of outlet tube 130. For example, outer surfaces of outlet tube 130 may fit against the inner surface of outlet channel 117, and the top (first) end 132 may be fixed to outlet channel 117 via various suitable means, such as an adhesive, friction fit, ridges/grooves, etc.

Outlet tube 130 may have a first opening at the top (first) end 132, and a second opening at the bottom (second) end 134. In some embodiments, the first and second openings of outlet tube 130 may have a diameter ranging from about 0.06 inches to about 0.10 inches. The bottom (second) end 134 may be in fluid communication with outlet 34 such that outlet channel 117 may be configured to deliver the agent stored in housing 102, and/or the pressurized fluid received from the pressurized fluid source, to catheter 36 via outlet tube 130 upon fluidizing the agent in cavity 101. Therefore, outlet channel 117 may define an outlet of cone assembly 110.

Still referring to FIG. 4, inlet channel 119 may be in fluid communication with outlet channel 117 via intermediate channel 118, and configured to receive at least a portion of fluid inlet 120. In the example, outlet channel 117 may have a longitudinal length defined between opposing ends that are arranged in a vertical orientation within cone assembly 110 (e.g., parallel to central axis A), extending beneath exit opening 114. Inlet channel 119 may have a longitudinal length defined between opposing ends that are arranged in a horizontal orientation (e.g., parallel to a central axis B of fluid inlet 120) that is perpendicular (or otherwise angled) to the vertical orientation of outlet channel 117 (and central axis A).

In the example, a diameter of inlet channel 119 may be sized relatively greater than a diameter of outlet channel 117. Further, inlet channel 119 may be sized, shaped, and configured to form a cavity and/or gap between the portion of fluid inlet 120 (received within inlet channel 119) and intermediate channel 118. Fluid inlet 120 may include an inlet port 122, an outlet port 124, and a channel 126 positioned between inlet port 122 and outlet port 124. Outlet port 124 may be sized, shaped, and configured to extend into inlet channel 119.

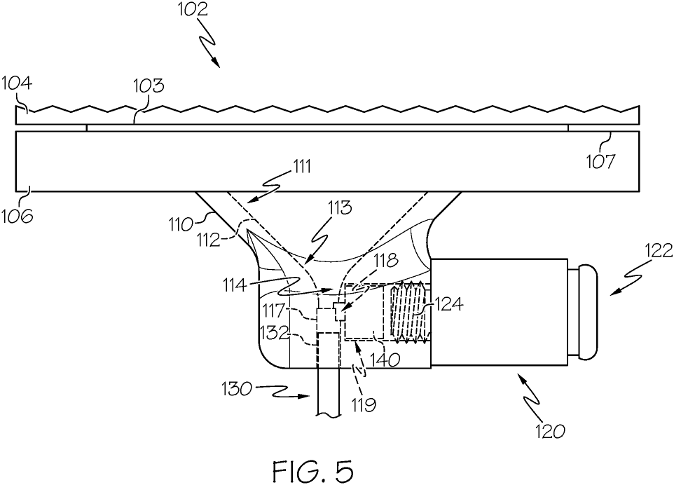

Still referring to FIG. 4, an exterior surface of outlet port 124 may include a fastening mechanism, such as a threaded portion, that is configured to mate with a corresponding fastening mechanism (e.g., a complimentary threaded portion) of inlet channel 119 to secure fluid inlet 120 to cone assembly 110. Inlet port 122 may be fluidly coupled to a tubing or other suitable component within delivery system 10 that is in fluid communication with the pressurized fluid source for delivering fluid to inlet channel 119. As described in detail below, cone assembly 110 may include / house a filter 140 (e.g., a sintered disc insert, see FIG. 5), and inlet channel 119 may be configured to receive filter 140 within the cavity and/or gap formed between intermediate channel 118 and fluid inlet 120.

Referring now to FIG. 5, filter 140 may be disposed within inlet channel 119. Filter 140 may be sized and shaped in accordance with a cross-sectional profile (e.g., diameter) of inlet channel 119, such that an exterior surface of filter 140 may be in contact with an interior surface of inlet channel 119. Thus, a pressurized fluid received within inlet channel 119 via fluid inlet 120 may be required to pass through filter 140 prior to entering intermediate channel 118 and outlet channel 117. Filter 140 may have a generally cylindrical shape that is sized to abut against an inner surface of inlet channel 119, such that the surfaces of filter 140 and inlet channel 119 may serve to form a boundary that only permit the passage of pressurized fluid through filter 140. As described further herein, filter 140 may be configured to inhibit the passage of the agent from housing 102 to fluid inlet 120. In some embodiments, filter 140 may be sealed with respect to inlet channel 119 (e.g., O-ring seal, substances such as adhesives, etc.) to inhibit the agent from passing between cavity 101 and fluid inlet 120. In the example, filter 140 may have a diameter ranging from about 0.15 inches to about 0.20 inches.

Filter 140 may have a constant thickness (i.e., a cross-sectional profile as measured along a longitudinal length of filter 140). Filter 140 may be sintered such that openings and tortuous paths / passages are formed between opposing ends of filter 140. The openings and tortuous passages may be sized such that the agent does not pass through the passages between a first end of filter 140 (e.g., adjacent to intermediate channel 118) and a second end of filter 140 (e.g., adjacent to fluid inlet 120). In the embodiment, filter 140 may include a sintered metal disc received within inlet channel 119. In other embodiments, filter 140 may be an injection-molded porous structure that is integral with inlet channel 119. In some embodiments of the latter, filter 140 and inlet channel 119 may form a unitary body.

Fluid from fluid inlet 120 may be permitted to flow through the openings in filter 140. In some examples, the openings may have sizes between approximately 2 microns to 200 microns, such as 40 microns to 100 microns. The agent stored in cavity 101 of housing 102 may include, for example, semi-cohesive materials, such as chitosan acetate. The agent may include particles that generally have a round shape with particle sizes ranging from approximately 320 microns to 500 microns, such that the agent may be too large to pass through the openings of filter 140. A density of the particles of the agent may be about 1.5 g/cm³.

In some embodiments, filter 140 may be formed via, for example, additive manufacturing techniques (e.g., three-dimensional printing). For example, a pattern or model created to form filter 140 may incorporate sintered openings and/or pores. Such openings may be formed in various suitable shapes, and may be substantially uniform or may vary. In other words, a size, shape, and/or distribution of the plurality of openings / pores in filter 140 may be substantially uniform relative to one another. In other embodiments, the plurality of openings / pores may have varying sizes, shapes, and/or spatial distribution relative to one another along and within filter 140. In some embodiments, filter 140 may be sintered, formed of a porous metal, formed of a lattice printed material, and more. By providing a sintered and/or porous filter, it should be appreciated that a fluidization consistency may be increased, and potential clogging caused by the agent may be reduced, as described further herein.

Filter 140 may be printed using, for example, powder bed fusion methods (e.g., via electron beam or laser beam). Alternatively, a solid model of filter 140 may be created, and energy may be applied to form pores (passages) in filter 140. For example, a laser may be applied to a combination of metal powder and a foaming agent, which results in the development of pores / passages through filter 140. Alternatively, a laser may be applied with energy calibrated so as to form pores as a metal powder is fused.

The tortuous passages of filter 140 may cause a pressurized fluid flowing through filter 140 (e.g., from fluid inlet 120) to enter intermediate channel 118 at a random velocity field, i.e. a wide variety of vectors, including angles, velocities, and/or pressures, at the same time. The fluid exiting filter 140 may have a turbulent flow pattern (e.g., a radial pattern). The varying vectors with which the pressurized fluid may enter intermediate channel 118, and ultimately a portion of cavity 101 storing the agent via exit opening 114, may cause the agent within housing 102 to become fluidized, e.g. have a consistency and/or flow that resembles a fluid. It should be appreciated that a size, shape, and/or location of the sintered / porous filter 140 may at least partially determine a delivery rate of the agent from delivery system 10, and a size, location, and/or shape of filter 140 may provide varying fluidization performance capabilities, such as an average delivery rate of the agent. As shown and described in further detail herein, intermediate channel 118 may be further configured to at least partially determine the delivery rate of the agent from delivery system 10.

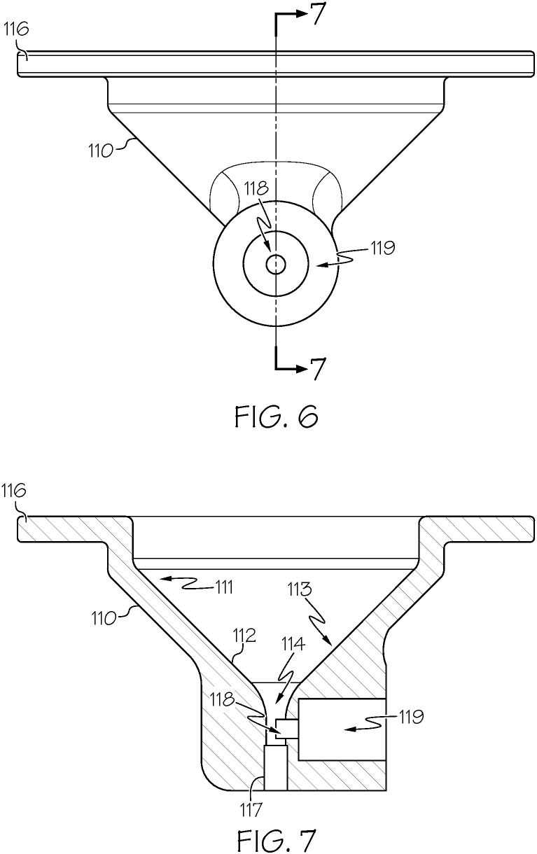

Referring to FIGS. 4-7, intermediate channel 118 may include a cut out formed within a body of cone assembly 110 between outlet channel 117 and inlet channel 119. Intermediate channel 118 may have a width as measured along central axis B, and a height as measured along central axis A, that define a cross-sectional passage for receiving the pressurized fluid from filter 140. Intermediate channel 118 may be configured to cause the pressurized fluid flowing from filter 140 to enter into a cavity defined by the inner wall 112 at an optimal velocity and/or pressure, thereby generating a turbulent flow of fluid that may cause the agent within housing 102 to become fluidized.

The turbulent flow of fluid (which may result in fluidization, such as a liquid sand effect, of the agent) may further aid in flowing the agent through exit opening 114 and outlet channel 117 while preventing or minimizing clogging of the agent as it is received in outlet tube 130. When fluidized, movement of the plurality of particles of the agent may resembles that of a liquid, such that the agent retains fluid-like qualities. Therefore, the turbulent flow of fluid may cause the agent to imitate the effect of a liquid sand moving freely without resistance. Fluidization may further aid in breaking up agglomerates of the agent prior to exiting cone assembly 110. Intermediate channel 118 may be configured to transfer the pressurized fluid into the cavity 101 to aerate the agent, and to deliver a mixture of the agent and fluid out of cone assembly 110 at an acceptable delivery rate. Accordingly, intermediate channel 118 may prevent clogging and deliver large volumes of the fluidized agent at an acceptable rate. In the example, intermediate channel 118 may be configured to generate a delivery rate of the fluidized agent (with a fluid flow rate of 5 SLPM) ranging from about 0.03 g/s to about 0.13 g/s, such as 0.1 g/s.

Referring specifically to FIGS. 5 and 7, intermediate channel 118 may include a cut out that is formed within cone assembly 110 with one or more interior walls and/or surfaces that are sized to at least partially extend into outlet channel 117. Stated differently, the walls and/or surfaces defining the cut out of intermediate channel 118 may have a longitudinal length extending parallel to central axis B, and that position intermediate channel 118 at least partially within a lumen of outlet channel 117. In some examples, outlet tube 130 may be positioned relative to outlet channel 117 with the top (first) end 132 positioned relatively beneath intermediate channel 118. In other examples, the top (first) end 132 may be positioned to at least partially overlap with intermediate channel 118.

A rate of delivering a fluidized agent from delivery system 10 may be at least partially based on a relative position of outlet tube 130 and intermediate channel 118. For example, cone assembly 110 may be configured to selectively adjust a delivery rate of the fluidized agent from delivery system 10 in accordance with a separation distance between the top end 132 (within outlet channel 117) and intermediate channel 118. In one embodiment, a delivery rate of the agent may increase as a distance (along the central axis A) between top end 132 and intermediate channel 118 increases. The distance between outlet tube 130 and intermediate channel 118 may range from about 0.10 inches to about 0.5 inches.

In exemplary use of delivery system 10, actuation mechanism 30 may be activated to permit the pressurized fluid (such as pressurized carbon dioxide) stored in the pressurized fluid source (such as a canister) to flow to fluid inlet 120. The fluid received at inlet port 122 may pass through channel 126 and transfer into inlet channel 119 via outlet port 124. The fluid may encounter the sintered filter 140 prior to being received by intermediate channel 118. Based on a size (e.g. diameter) and/or longitudinal length of intermediate channel 118, an angle, velocity, and/or pressure of the pressurized fluid may be controlled to deliver the fluid into the cavity defined by inner wall 112 with a turbulent flow pattern (e.g., a radial pattern). Accordingly, a random velocity field may be formed within cone assembly 110, and particularly the cavity 101 storing the agent. It should be appreciated that the pressurized fluid may be delivered to the agent stored adjacent to inner wall 112 by passing through outlet channel 117 and exit opening 114, respectively.

As the agent is urged by the pressurized fluid to move downward, it may encounter portions of inner wall 112 that vary in diameter, which may reduce clogging of the agent within cone assembly 110. The agent may be prone to bridging, which may result in clogging absent the variations in diameter of inner wall 112.

As described above, at the top (first) end 111, an inner surface of inner wall 112 may taper inward, moving in a downward direction. In some embodiments, an angle of inner wall 112 (relative to central axis A) may be greater than (i.e., steeper than) an angle of repose of the agent. An angle of repose of the agent may be an angle formed by a cone-like pile of the agent as it flows within cone assembly 110 and collects on a surface in the cone-like pile. The angle of repose may be the angle between a surface of inner wall 112 onto which the agent flows and a surface of the cone-like pile. The angle of repose may be related to friction or resistance to movements between particles of the agent. Since inner wall 112 may have an angle greater than the angle of repose of the agent, the agent may flow freely through exit opening 114 due to a force of gravity.

As best seen in FIG. 7, a top end of exit opening 114 may be wider than a bottom end of exit opening 114 adjacent to outlet channel 117. In this instance, the inner surface of exit opening 114 may taper radially inward toward central axis A and in a downward direction such that a size of exit opening 114 decreases. Alternatively, the top end of exit opening 114 may have substantially the same diameter as the bottom end. In this instance, exit opening 114 may have a constant or substantially constant diameter. In either instance, a bottom end of exit opening 114 may have a size that is the same as or substantially the same as the size of outlet channel 117 adjacent to the bottom end of exit opening 114. In passing through cone assembly 110, the agent may travel through constantly narrowing portions of inner wall 112 and exit opening 114, which may result in decreased clogging of the agent. Portions of inner wall 112 that are tapering and/or angled may have an angle of approximately 45 degrees relative to central axis A.

Upon moving (e.g., aerating, agitating, fluidizing) the agent in cone assembly 110 and/or housing 102, the fluidized agent may exit cone assembly 110 by flowing through exit opening 114 and outlet channel 117, and into outlet tube 130. The fluidized agent may be transferred by outlet tube 130 toward outlet 34 and into catheter 36 for delivery to a target treatment site. As the fluidized agent travels through outlet channel 117 and into outlet tube 130, at least a portion of the fluidized agent may be received within intermediate channel 118. However, the fluidized agent may not flow into inlet channel 119 and toward fluid inlet 120 due to filter 140. As described in detail above, filter 140 may be configured to inhibit the fluidized agent from passing through the sintered filter 140.

Dispensing portion 100 may provide numerous benefits in certain embodiments. For example, filter 140 can provide for a turbulent flow of fluid, and intermediate channel 118 may control a delivery rate of the turbulent flow of fluid as it combines with the agent. The turbulent flow of fluid delivered at the controlled rate may provide improvements to flow of the agent through outlet 34 as compared to devices without filter 140 and/or intermediate channel 118 that do not allow for controlling a delivery rate of a turbulent flow. The configuration of cone assembly 110, and particularly inner wall 112 and exit opening 114, may further facilitate delivery of the agent without clogging (or without significant clogging) due to, for example, bridging of the agent adjacent to outlet channel 117. Fluidization of the agent may have various advantages, including aerating the agent, reducing friction between particles of the agent, suspending particles of the agent in fluid (e.g., air or carbon dioxide) to propel them, faster delivery of particles of the agent, and/or delivery of the agent using less fluid. Fluidization may break up agglomerates of the agent.

While principles of this disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A device for delivering an agent, comprising:
a housing configured to store an agent;
a cone assembly at least partially received within the housing, the cone assembly including:
an inner wall configured to be adjacent to the agent;
an outlet channel extending from the inner wall;
an inlet channel; and
an intermediate channel between the outlet channel and the inlet channel, such that the inlet channel is in fluid communication with the outlet channel via the intermediate channel;
a filter disposed within the inlet channel and positioned adjacent to the intermediate channel, the filter including a plurality of pores configured to permit a fluid received by the inlet channel to pass through the intermediate channel to mix with the agent adjacent to the inner wall.

2. The device of claim 1, wherein the outlet channel is coupled to an outlet tube, such that the outlet tube is in fluid communication with the agent via the outlet channel, and the inlet channel is coupled to an inlet tube, such that the inlet tube is in fluid communication with the agent via the inlet channel.

3. The device of claim 2, wherein the inlet tube is configured to supply the fluid to the intermediate channel and through the filter for mixing with the agent.

4. The device of claim 3, wherein the outlet tube is configured to receive the agent from the inner wall via the outlet channel when the agent is moved by the fluid supplied from the inlet tube.

5. The device of claim 3, wherein the outlet tube includes a first end disposed within the outlet channel and positioned relatively underneath the intermediate channel.

6. The device of claim 3, wherein the outlet tube includes a first end disposed within the outlet channel and positioned relative to the intermediate channel such that the first end overlaps with the intermediate channel.

7. The device of claim 1, wherein the filter includes an insert received within the inlet channel.

8. The device of claim 1, wherein the filter is sintered, includes a porous metal, or includes a lattice printed material.

9. The device of claim 1, wherein the plurality of pores extend along tortuous paths between opposing ends of the filter.

10. The device of claim 1, wherein the cone assembly includes an opening at a first end of the cone assembly, and a diameter of space defined by an exterior surface of the inner wall decreases in a direction toward the opening at the first end.

11. The device of claim 10, wherein the intermediate channel is positioned relatively underneath the opening, and extends at least partially within the outlet channel and the inlet channel.

12. The device of claim 1, wherein the filter is coupled to the cone assembly within the inlet channel, such that the filter is removable from the inlet channel.

13. The device of claim 1, wherein the filter is integrally formed with the cone assembly.

14. The device of claim 1, wherein the housing includes a first body and a second body that are configured to engage the cone assembly along an outer rim of the cone assembly.

15. The device of claim 1, wherein the agent is a powder having a particle diameter between 320 microns and 500 microns, and the plurality of pores have a diameter between 40 microns and 100 microns.

16. A device for delivering an agent, comprising:
a housing configured to store an agent;
a cone assembly coupled to the housing, and configured to receive the agent, wherein the cone assembly includes a wall configured to be adjacent to the agent, and an intermediate channel positioned relatively below the wall at an end of the wall opposite an end of the wall that receives the agent, for controlling a flow of fluid toward the agent; and
a filter coupled to the cone assembly alongside the intermediate channel, the filter includes a plurality of pores configured such that the flow of fluid is allowed to pass through the intermediate channel via the filter and into a cavity defined by an inner surface of the wall to agitate the agent within the cone assembly.

17. The device of claim 16, wherein the filter is positioned such that the flow of fluid is permitted to pass through the intermediate channel only after passing through the plurality of pores, and the plurality of pores are configured such that the agent is not permitted to pass through the filter.

18. The device of claim 16, wherein the housing includes a first body and a second body that are configured to engage an outer rim of the cone assembly to securely couple the cone assembly to the housing.

19. The device of claim 16, wherein the filter is sintered, includes a porous metal, or includes a lattice printed material.

20. A device for delivering an agent, comprising:
a housing configured to hold an agent;
a cone coupled to the housing for funneling the agent toward an opening, the cone includes an outlet in fluid communication with the opening, an inlet in fluid communication with the outlet, and an intermediate channel between the outlet and the inlet; and
a sintered filter disposed within the inlet and positioned adjacent to the intermediate channel, the sintered filter including a plurality of pores that are configured such that gas is permitted to pass through the filter and into the cone via the intermediate channel to move the agent within the housing, wherein the intermediate channel is sized to control a flow of the gas into the cone for moving the agent.
